(19) United States
(12) Patent Application Publication
(10) Pub. No.: US 2015/0057535 A1
(43) Pub. Date: Feb. 26, 2015

(54) SYSTEMS AND METHODS FOR ATTENUATION CORRECTION IN TIME-OF-FLIGHT POSITRON EMISSION TOMOGRAPHY

(71) Applicant: Arkadiusz Sitek, Ashland, MA (US)

(72) Inventor: Arkadiusz Sitek, Ashland, MA (US)

(21) Appl. No.: 14/503,812

(22) Filed: Oct. 1, 2014

Related U.S. Application Data
(60) Provisional application No. 61/855,144, filed on May 8, 2013.

(51) Int. Cl. A61B 6/00; G06T 11/00; A61B 6/03

(52) U.S. Cl. CPC A61B 6/5205; A61B 6/037; A61B 6/481; A61B 6/4233; G06T 11/003; G06T 2207/10104
USPC 600/426; 382/131

(57) ABSTRACT

Systems and methods for attenuation correction in time-of-flight (“TOF”) positron emission tomography (“PET”) imaging are provided. In some aspects, a provided PET system includes a plurality of detectors arranged about a bore configured to receive the subject, the plurality of detectors configured to acquire gamma rays emitted from the subject as a result of a radiotracer administered to the subject. The system also includes a data acquisition processor in communication with the plurality of detectors and configured to acquire TOF-PET data corresponding to acquired gamma rays. The system further includes an image reconstruction processor configured to process the acquired TOF-PET data with steps including estimating from the TOF-PET data, a posterior probability distribution of attenuation-corrected TOF-PET data, and reconstructing attenuation-corrected images from the posterior probability distribution of attenuation-corrected TOF-PET data. In some aspects, the image reconstruction processor is further configured to produce attenuation maps using the attenuation-corrected TOF-PET data.

### CROSS-REFERENCE TO RELATED APPLICATIONS

This application claims the benefit of U.S. Provisional Patent Application Ser. No. 61/885,144, filed on Oct. 1, 2013, and entitled “SYSTEMS AND METHODS FOR ATTENUATION CORRECTION IN TIME-OF-FLIGHT POSITRON EMISSION TOMOGRAPHY.”

### STATEMENT REGARDING FEDERALLY SPONSORED RESEARCH

This invention was made with government support under HL06474 awarded by the National Institutes of Health. The government has certain rights in the invention.

### BACKGROUND OF THE INVENTION

The field of the invention is systems and methods for medical and molecular imaging. More particularly, the invention relates to systems and methods for performing attenuation correction for time-of-flight (“TOF”) positron emission tomography (“PET”) imaging.

Positrons are positively charged electrons that are emitted by radionuclides that have been prepared using a cyclotron or other device. Radionuclides are generally employed as radioactive tracers in medical imaging by being incorporated into various substances, such as glucose or carbon dioxide, and administered to a patient. The activity of such “radiopharmaceuticals” can then be monitored to provide information related to biochemical or physiological processes, such as blood flow, fatty acid and glucose metabolism, and protein synthesis, for uses including identifying tissue function abnormalities or tumors.

The most common clinical applications of PET imaging is in oncology and cardiology for detecting and staging of cancer and cardiac diseases, as well as for monitoring treatment response. In a PET imaging scan, a radioactive tracer that emits positrons is administered to the body of a patient. The released positrons immediately annihilate, creating photon pairs with 511 keV energies that propagate in opposite directions from the annihilation point. The volume distribution and concentration of radioactive tracers in the body is determined based on the detection of radiation outside the patient.

A PET scanner includes one or more rings of detectors that encircle the patient and convert the energy of each 511 keV photon into a flash of light that is sensed by a radiation detector, such as a photomultiplier tube (“PMT”). Coincidence detection circuits connected to the radiation detectors record only those photons that are detected simultaneously by two detectors located on opposite sides of the patient The number of such simultaneous events indicates the number of positron annihilations that occurred along a virtual line joining the two opposing detectors, called the line of response (“LOR”). An image indicative of the tissue concentration of the positron emitting radionuclide is created by determining the number of such annihilation events at each location within the field-of-view.

New generation scanners are equipped with a time-of-flight (“TOF”) capability that enables measurements of the difference in arrival time of the released photons. This information is used to pinpoint the actual location of the annihilation on the LOR. Before reaching the detectors, however, photons may be stopped or attenuated by tissues in a patient's body, leading to artifacts in the resulting volumetric images and incorrect measurement of tracer concentration. This occurs due to photon interactions with atoms in different body tissues, where scattered photons experiencing energy loss may be absorbed by the atoms or may escape with reduced energy and subsequently be rejected by the PET scanner. In this regard, attenuation correction is essential for TOF-PET imaging since absent appropriate correction, significant and region-specific errors would occur in reconstructed PET images, in dependence of the various tissue attenuation properties.

To correct for attenuation, a number of techniques have been previously implemented, including either directly measuring, or calculating attenuation tissue properties. For instance, one approach involves pre-correction of measured positron emission data using attenuation factors, for instance, derived from transmission scans or derived from forward projecting determined attenuation maps, representing the spatial distribution of the attenuation coefficients. Specifically, obtained attenuation factors are used to modify sinograms that contain the number of annihilation events at each location within a field-of-view.

In general, attenuation maps are generated using x-ray attenuation measurements obtained using hybrid PET-computed tomography (“CT”) scanners or derived from magnetic resonance (“MR”) images from PET/MR scanners. However, in addition to the added complexities and cost of multi-modality scanning, attenuation maps obtained using CT or MR images carry serious risks of artifacts in the attenuation-corrected PET image. Specifically, such artifacts may be inherent to CT or MR imaging, or they may be due to geometrical misalignment between PET and CT or MRI images, for instance, as a result of patient movement. Specifically with regard to MRI, additional errors from the transformation of MRI image values to values of attenuation coefficients also present a serious drawback. For instance, MR signals are dependent upon proton densities and relaxation properties of tissues, and are not explicitly related to attenuation of ionizing radiation.

Therefore, given at least the drawbacks described above, there is a need for improved imaging technologies, and specifically technologies related to TOF-PET imaging capable of robust photon attenuation correction without shortcomings associated with performing x-ray transmission or magnetic resonance image measurements.

### SUMMARY OF THE INVENTION

The present disclosure overcomes drawbacks of prior technologies by providing a novel approach for correcting errors due to photon attenuation. In particular, disclosed systems and methods are directed to time-of-flight (“TOF”) positron emission tomography (“PET”) imaging.

Among various features and capabilities, provided systems and methods, in accordance with various aspects of the present disclosure, can directly utilize TOF-PET data acquired with a PET system to generate photon attenuation maps as well as produce attenuation-corrected images. Specifically, a posterior probability distribution of attenuation values may be estimated from the acquired TOF-PET data. For example, a Markov Chain Monte Carlo technique may be used to estimate sample values of the posterior, although other approaches may also be possible. From the estimates of the posterior, an attenuation map can then be produced by computing one of the mean, the marginalized median, and the marginalized maximum values of the posterior. As an additional feature, the posterior probability distribution of attenuation-corrected activity values can be estimated, from which an attenuation-corrected image can be directly produced.

In accordance with one aspect of the present disclosure, a method for reconstructing an attenuation-corrected image using a time-of-flight (“TOF”) positron emission tomography (“PET”) system is provided. The method includes acquiring TOF-PET data using the TOF-PET system and estimating from the TOF-PET data, a posterior probability distribution of attenuation-corrected TOF-PET data. The method also includes reconstructing an attenuation-corrected image from the posterior probability distribution of attenuation-corrected TOF-PET data.

In accordance with another aspect of the present disclosure, a method for producing an attenuation map from time-of-flight (“TOF”) positron emission tomography (“PET”) data acquired with a PET system is provided. The method includes acquiring TOF-PET data using the PET system, and estimating from the TOF-PET data, a posterior probability distribution of attenuation values. The method also includes producing an attenuation map from the posterior probability distribution of attenuation values.

In accordance with yet another aspect of the present disclosure, a positron emission tomography (“PET”) system for producing attenuation-corrected images of a subject is provided. The system includes a plurality of detectors arranged about a bore configured to receive the subject, the plurality of detectors configured to acquire gamma rays emitted from the subject as a result of a radiotracer administered to the subject. The system also includes a data acquisition processor in communication with the plurality of detectors and configured to acquire TOF-PET data corresponding to acquired gamma rays. The system further includes an image reconstruction processor configured to process the acquired TOF-PET data with steps including estimating from the TOF-PET data, a posterior probability distribution of attenuation-corrected TOF-PET data, and reconstructing an attenuation-corrected image from the posterior probability distribution of attenuation-corrected TOF-PET data.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flowchart setting forth the steps of an example of a method for reconstructing attenuation-corrected images and/or attenuation maps from time-of-flight positron emission tomography (“TOF-PET”) data.

FIG. 2 is a flowchart setting forth the steps of an example of a method for estimating posterior probabilities of quantities of interest in TOF-PET data, such as the number of counts per voxel and the attenuation coefficients for each voxel.

FIG. 3 is a schematic diagram of a PET imaging system in accordance with aspects of the present disclosure.

### DETAILED DESCRIPTION OF THE INVENTION

Described herein is a novel approach for reconstructing attenuation corrected time-of-flight (“TOF”) positron emission tomography (“PET”) images, and alternatively reconstructing attenuation maps directly from the TOF-PET data. Specifically, the need for performing transmission scans or use of magnetic resonance images for attenuation correction in TOF-PET imaging is removed. As will be described, attenuation correction maps can be determined based on emission data, which eliminates sources of serious artifacts introduced by using attenuation maps derived from computed tomography (“CT”) or magnetic resonance imaging (“MRI”).

The techniques described herein employ the concept of origin ensembles based on Bayesian statistics, whereby the number of detected counts per voxel and attenuation factors in the sinogram domain are the quantities of interest used to generate origin ensemble attenuation maps estimation (“OEAME”). This allows a highly efficient implementation of correction for attenuation without use of corrections based on separate CT transmission or MRI scans. With an increasingly larger fraction of new PET scanners having TOF capability, the methods described herein provide a robust and efficient approach for providing attenuation correction in TOF-PET imaging.

Specifically, the equation for the posterior probability of complete data given observed incomplete data may be given by,

$$p ( y \vert g ; a ) = \frac{1}{Z} {\prod }_{i = 1}^{I} \frac{{c}_{i} !}{{\tilde{ɛ}}_{i}^{{c}_{i}}} {\prod }_{k , t = 1}^{K , T} \frac{{( {\tilde{α}}_{kti} )}^{{y}_{kti}}}{{y}_{kti} !} for y ∈ {Y}_{g} ; \qquad ( 1 )$$

where \tilde{α}ₖₜᵢ=aₖαₖₜᵢ, with aₖ being the attenuation factor of the k^{th} projection bin and αₖₜᵢ containing all the factors, other than attenuation, that affect the probability of detection of counts emitted in the i^{th} image voxel and detected in the k^{th} bin and t^{th} TOF-bin.

The quantity, a, can be referred to as an attenuation-factor sinogram to differentiate it from the attenuation sinogram, which is an integral through the attenuation coefficient of the object along the LORs. There is a simple relation between the attenuation and attenuation-factor sinograms, as the elements of the attenuation-factor sinogram are obtained by taking the inverse of the exponent of the elements of the attenuation sinogram.

Because elements of the attenuation sinogram are positive, the values of the attenuation-factor sinogram, a, are in the range [0,1]. The element, αₖₜᵢ, describes the probability that an event is emitted in the i^{th} image voxel and detected in the k^{th} bin and the t^{th} time-of-flight bin (TOF-bin) if there is no attenuation.

An assumption is made that the attenuation is the same for all TOF-bins detected in the same bin, k. The voxel sensitivity, \tilde{ε}ᵢ, for the i^{th} image voxel is defined as,

$${\tilde{ɛ}}_{i} = {\sum }_{k , t = 1}^{K , T} {\tilde{α}}_{kti} . \qquad ( 2 )$$

The value of yₖₜᵢ is the number of events emitted in the i^{th} voxel and detected in {k,t}. The total number of emitted and detected counts from the i^{th} voxel is denoted by,

$${c}_{i} = {\sum }_{k , t = 1}^{K , T} {y}_{kti} . \qquad ( 3 )$$

The condition in Eqn. (1) that y ε Y_{g} signifies that the posterior is non-zero only for y such that,

$${\sum }_{i = 1}^{I} {y}_{kti} = {g}_{kt} ; \qquad ( 4 )$$

where gₖₜ is an element of the data vector, g.

The posterior in Eqn. (1) is a function of a since the sensitivity, \tilde{ε}, is a function of the attenuation-factor sinogram, a. If a is considered as a random variable and assigned a uniform prior equal to 1 for αₖε [0,1] and zero otherwise, the posterior in Eqn. (1) can be rewritten as a conditional joint probability, such as

p(y,a|g)∝p(y|g;a) (5);
where the conditional statistical independence of y and a is assumed. The posterior of a is obtained by marginalization of y, yielding the following:

$$\begin{matrix}\begin{matrix}p ( a \vert g ) = {\sum }_{y ∈ {Y}_{g}} p ( y , a \vert g ) \\ = \frac{1}{Z} {\sum }_{y ∈ Yg} {\prod }_{i = 1}^{I} \frac{{c}_{i} !}{{\tilde{ɛ}}_{i}^{{c}_{i}}} {\prod }_{k = 1}^{K} \frac{{( {a}_{k} {α}_{kti} )}^{{y}_{kti}}}{{y}_{kti} !} for {a}_{k} ∈ [ 0 , 1 ] \\ = \frac{{Z}_{y}}{Z} ( {\prod }_{k = 1}^{K} {a}_{k}^{{g}_{k}} ) ( {\prod }_{i = 1}^{I} \frac{1}{{\tilde{ɛ}}_{i}^{{c}_{i}}} ) ;\end{matrix} \\ where , & ( 6 ) \\ {g}_{k} {\sum }_{i , t = 1}^{I , T} {y}_{kti} ; & ( 7 )\end{matrix}$$

which is the total number of counts detected in the k^{th} bin (i.e., the sum of all TOF bins) and Z_{y} is constant independent of a. An interesting observation can be made considering the above posterior, as it is independent on the operation of scaling of a. In other words,

p(a|g)=p(ba|g) (8);
where b is any non-zero scaling constant such that for all k, baₖε [0,1].

Using the posterior in Eqn. (6), the minimum mean square error (“MMSE”) estimator of can be obtained as follows:

$$\hat{a} = {\int }_{0}^{1} d {a}_{1} … {\int }_{0}^{1} d {a}_{k} a {\sum }_{y ∈ {Y}_{g}} p ( y , a \vert g ) . \qquad ( 9 )$$

The calculation of the estimator in Eqn. (9) can be done using Markov Chain Monte Carlo (“MCMC”) methods with Metropolis-Hastings sampling. It will be appreciated that other MMSE estimates (e.g., ĉ_{MMSE}) can be easily obtained without additional computing cost by performing the MC calculations. Note that the component-wise division ĉ_{MMSE}/\tilde{ε} is the estimate of attenuation-corrected activity; therefore, the method described herein provides all the necessary information for full tomographic reconstruction. In fact, the estimator of â need not be used in clinic; instead, ĉ_{MMSE}/\tilde{ε} can be calculated directly using the appropriate estimator, such as,

$${\hat{c}}_{MMSE} = {\sum }_{y ∈ {Y}_{g}} c {\int }_{0}^{1} d {a}_{1} … {\int }_{0}^{1} d {a}_{k} p ( y , a \vert g ) ; \qquad ( 10 )$$

where again, c, is a vector containing the number of emitted events per voxel.

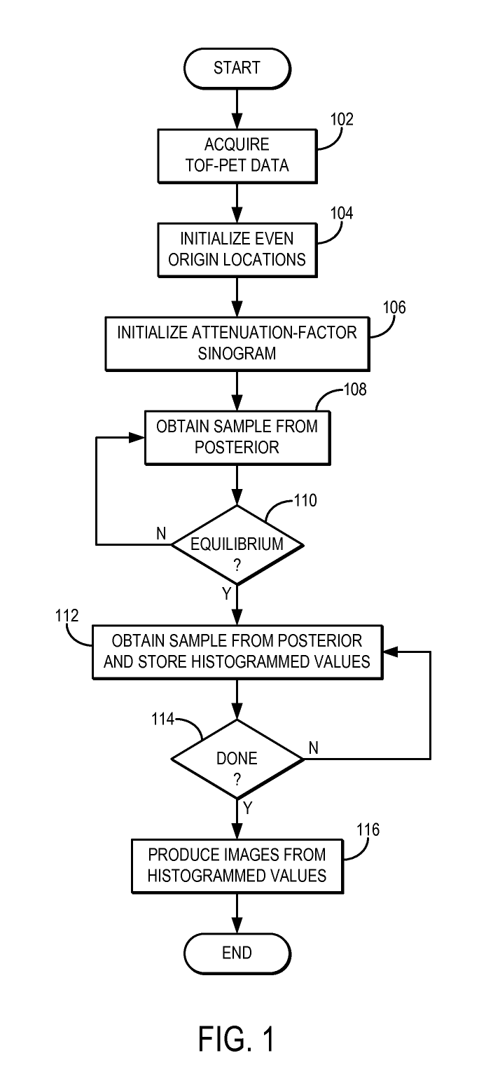

Using the techniques described here, an example of a method for image reconstruction with attenuation correction without transmission measurement can be summarized as follows. Referring now to FIG. 1, a flowchart setting forth the steps of an example of a method for reconstructing an attenuation-corrected image from TOF PET imaging data is illustrated. The method begins with acquiring TOF PET data using a PET imaging system, as indicated at step 102.

The reconstruction process begins with initializing a first estimate of the locations for the origins of all the detected events, as indicated at step 104. By way of example, event origin locations can be estimated by assigning them to voxels at the most likely location of occurrence based on the corresponding LOR and measured TOF difference. An attenuation-factor sinogram, a , is the initialized, as indicated at step 106. For instance, the elements of the attenuation-factor sinogram can be initialized to values of 1, which corresponds to zero attenuation. As indicated at step 108, the next step in the reconstruction is to obtain samples of the posterior probability of complete data, such as the posterior provided above in Eqn. (6).

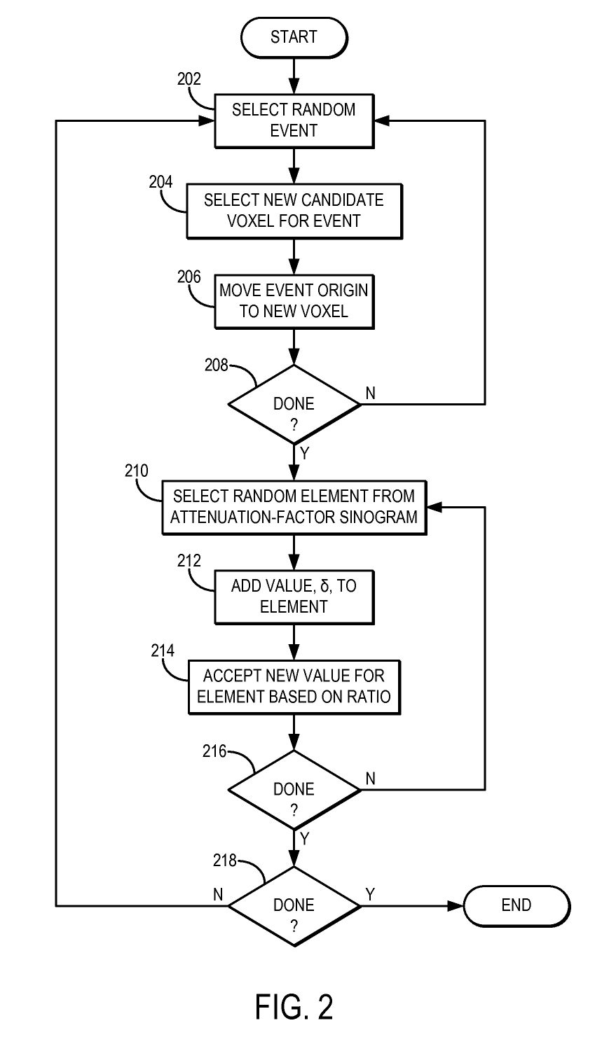

The process of obtaining samples from this posterior distribution proceeds as shown in the flowchart illustrated in FIG. 2. First, an event is randomly selected, as indicated at step 202. In order to increase efficiency of the algorithm and to reduce the bias due to the approximation, only events with origins inside the object are selected. Next, a new candidate voxel, i′, for the selected event is randomly selected, as indicated at step 204. Then, as indicated at step 206, the origin of the selected event is then stochastically moved to the new candidate voxel with a transition probability equal to,

$$min ( 1 , \frac{{{α}_{{ni}^{′}} ( {c}_{si} - 1 )}^{{c}_{si} - 1} {( {c}_{{si}^{′}} + 1 )}^{{c}_{{si}^{′ + 1}}} / {ɛ}_{{i}^{′}}}{{{α}_{ni} ( {c}_{si} )}^{{c}_{si}} {( {c}_{{si}^{′}} )}^{{c}_{{si}^{′}}} / {ɛ}_{i}} ) ; \qquad ( 11 )$$

where n is an index indicating the n^{th} detected event, with n=1, . . . ,N. The specification of event origin locations for all N events defines the state, s, in the ensemble of all I^{N} states (i.e., the origin ensemble). The number of event origins in the i^{th} voxel is denoted as cₛᵢ.

A decision is then made at decision block 208 whether additional iterations of steps 202-206 should be performed. This decision can be assessed by checking whether a stopping criterion has been satisfied, or by enforcing a predetermined number of iterations. As an example, the number of iterations, A, to be performed can be approximately equal to the number of detected events, N.

Next, a random element, aₖ, from the attenuation-factor sinogram, a, is selected, as indicated at step 210. A new value for the selected element, a′ₖ, is then generated by modifying the selected element, aₖ, by a randomly selected value, δ, that is drawn from a uniform distribution, [−γ, γ], as indicated at step 212. Selection of the value of γ does not affect the result of this method, but it does affect the computational speed; however, the random value, δ, is selected such that a′ₖε [0,1].

As indicated at step 214, the new value, a′ₖ, is accepted stochastically with an acceptance probability of,

$$min ( 1 , \frac{p ( {a}^{′} , y \vert g )}{p ( a , y \vert g )} ) ; \qquad ( 12 )$$

where, using Eqn. (6),

$$\frac{p ( {a}^{′} , y \vert g )}{p ( a , y \vert g )} = {( 1 + \frac{δ}{{a}_{k}} )}^{{g}_{k}} {\prod }_{i = 1}^{I} {( 1 + \frac{{α}_{ki} δ}{{\tilde{ɛ}}_{i}} )}^{- {c}_{i}} . \qquad ( 13 )$$

A decision is then made at decision block 216 whether additional iterations of the steps 210-214 should be performed. This decision can be assessed by checking whether a stopping criterion has been satisfied, or by enforcing a predetermined number of iterations. As an example, the number of iterations, B, to be performed can be approximately equal to a percentage of the number of detected events, such as one or ten percent.

A decision is made at decision block 218 whether the algorithm terminates or whether steps 202-216 should be repeated. This decision can be assessed by checking whether a stopping criterion has been satisfied, or by enforcing a predetermined number of iterations for the algorithm.

Referring again to FIG. 1, at decision block 110, the convergence to equilibrium is assessed and a determination as to whether to continue iterating step 108 is made. Once in equilibrium, step 108 will generate states with frequencies proportional to the probabilities of these states defined by the posterior in Eqn. (6), thereby effectively calculating ensemble expectation values using a Monte Carlo approach. By way of example, convergence to equilibrium can be assessed by checking whether a stopping criterion has been satisfied, or by enforcing a predetermined number of iterations.

The result is independent of A, B, and δ, but the convergence to equilibrium (i.e., the computation time) may be affected by the values of these parameters. As noted above, A typically will be on the order of the number of events detected, and B will typically be some percentage of A, such as one or ten percent. The total number of loops for the burn-in period (i.e., before the Markov chain reaches equilibration) can be around 500-1000, and the total number of measurement loops can be around 1000-100,000 depending on the quantity of interest. As an example, if only a point estimate is sought during image reconstruction, the total number of measurement loops will be on the order of around 1000.

When equilibrium is reached, the posterior is sampled, as indicated in step 112, similar to the process described above for step 108. The value of each quantity of interest (e.g., y and a) is also histogrammed in each iteration of step 112 to serve as approximation of the marginalized posterior distribution of that value. A determination is then made at decision block 114 whether enough samples of the marginalized posterior distribution(s) have been obtained. If not, then step 112 is repeated again; otherwise, an estimate of the reconstructed image is produced from the histogrammed values, as indicated at step 116. As an example, an estimate of the image can be generated by computing the mean, median, or maximum of the histogram corresponding to total number of origins in each voxel, and then dividing that value by voxel sensitivity. As described above, images reconstructed in this manner are inherently corrected for the effects of the unknown attenuation maps. Alternatively, attenuation maps can be generated using the appropriate estimator, as also described above.

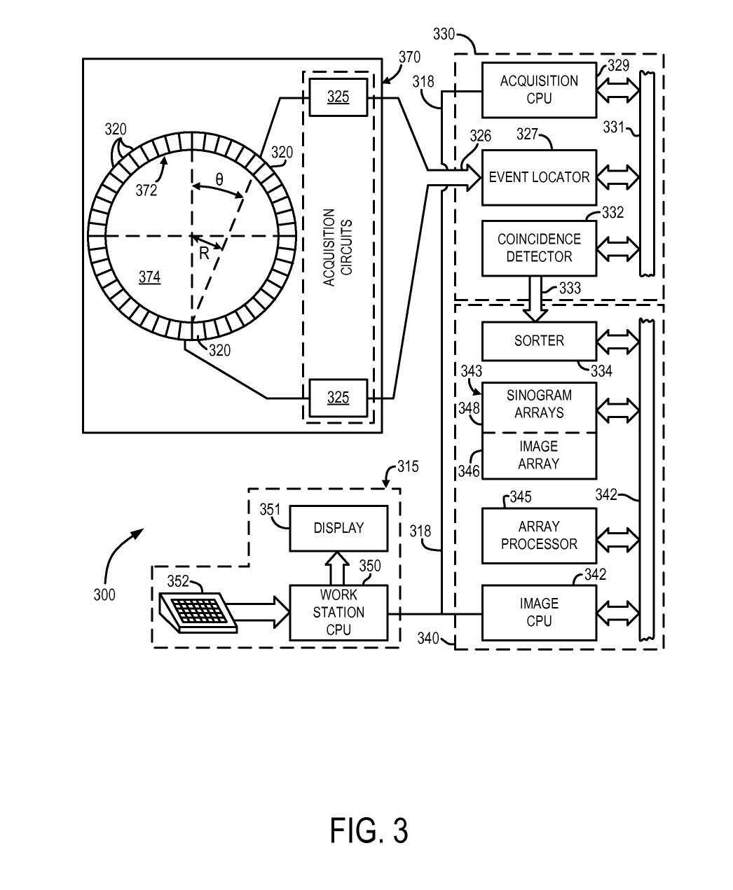

Referring particularly to FIG. 3, a schematic diagram of a PET system 300 is shown. Although PET system 300, as represented in the example of FIG. 3, can be implemented as a stand-alone imaging system, in accordance with some aspects of the present disclosure, it may be appreciated that PET system 300 may also utilized in combination with other imaging systems. For example, PET system 300 may be integrated into a multi-modality, or hybrid, imaging system, such as a PET/CT system, or a PET/MR system. In some aspects, raw or processed PET data, or images, generated using PET system 300 may be directly used to generate photon attenuation maps, and/or attenuation-corrected images. In other aspects, raw or processed PET data, or images, may be combined with information from other raw or processed data or images, such as CT or MR data or images, to generate photon attenuation maps, and/or attenuation-corrected images.

As illustrated in FIG. 3, PET system 300 includes a gantry 370, which supports a detector ring assembly 372. The detector ring 372 includes detector units 320. The signals produced by the detector units 320 are then received by a set of acquisition circuits 325, which produce digital signals indicating the line of response and the total energy. These signals are sent through a communications link 326 to an event locator circuit 327. Each acquisition circuit 325 also produces an event detection pulse (“EDP”) which indicates the exact moment the scintillation event took place.

The event locator circuits 327 form part of a data acquisition processor 330, which periodically samples the signals produced by the acquisition circuits 325. The processor 330 has an acquisition CPU 329 which controls communications on local area network 318 and a backplane bus 331. The event locator circuits 327 assemble the information regarding each valid event into a set of digital numbers that indicate precisely when the event took place and the position of the scintillator crystal which detected the event. This event data packet is conveyed to a coincidence detector 332 which is also part of the data acquisition processor 330.

The coincidence detector 332 accepts the event data packets from the event locators 327 and determines if any two of them are in coincidence. Coincidence is determined by a number of factors. First, the time markers in each event data packet must be within a preset time of each other, and second, the locations indicated by the two event data packets must lie on a straight line. Events that cannot be paired are discarded, but coincident event pairs are located and recorded as a coincidence data packet.

The coincidence data packets are conveyed through a link 333 to a sorter 334 where they are used to form a sinogram. The sorter 334 forms part of an image reconstruction processor 340. The sorter 334 counts all events occurring along each projection ray (R, θ) and organizes them into a two dimensional sinogram array 348 which is stored in a memory module 343. In other words, a count at sinogram location (R, θ) is increased each time a coincidence data packet at that projection ray is received.

The image reconstruction processor 340 also includes an image CPU 342 that controls a backplane bus 341 and links it to the local area network 318. An array processor 345 also connects to the backplane 341 and it reconstructs an image from the sinogram array 348. The resulting image array 346 is stored in memory module 343 and is output by the image CPU 342 to the operator work station 315.

The image reconstruction processor 340 may be configured to process acquired TOF-PET data by estimating from the TOF-PET data, in accordance with aspects of the present disclosure, a posterior probability distribution of attenuation-corrected TOF-PET data. For example, the image reconstruction processor 340 may obtain sample values of the posterior probability distribution by applying a Markov Chain Monte Carlo technique.

In some aspects, the image reconstruction processor 340 may is configured to produce one or more attenuation maps by computing one of a mean of the posterior probability distribution, or a marginalized median of the posterior probability distribution, or a marginalized maximum of the posterior probability distribution. In other aspects, the image reconstruction processor 340 may be configured to reconstruct attenuation-corrected images from the posterior probability distribution of attenuation-corrected TOF-PET data. Specifically, the image reconstruction processor 340 is configured to reconstruct images having voxels that contain attenuation-corrected estimates of a number of emissions per voxel, and produce from these images, images having voxels that contain attenuation-corrected estimates of activity.

The operator work station 315 includes a CPU 350, a display 351 and a keyboard 352. The CPU 350 connects to the network 218 and it scans the keyboard 252 for input information. Through the keyboard 352 and associated control panel switches, the operator can control the calibration of the PET scanner and its configuration. Similarly, the operator can control the display of the resulting image on the display 351 and perform image enhancement functions using programs executed by the work station CPU 350.

The present invention has been described in terms of one or more preferred embodiments, and it should be appreciated that many equivalents, alternatives, variations, and modifications, aside from those expressly stated, are possible and within the scope of the invention.

1. A method for reconstructing an attenuation-corrected image using a time-of-flight (“TOF”) positron emission tomography (“PET”) system, the steps of the method comprising:
a) acquiring TOF-PET data using the TOF-PET system;
b) estimating from the TOF-PET data, a posterior probability distribution of attenuation-corrected TOF-PET data; and
c) reconstructing an attenuation-corrected image from the posterior probability distribution of attenuation-corrected TOF-PET data.

2. The method as recited in claim 1 wherein step c) includes reconstructing the image by computing a mean of the posterior probability distribution.

3. The method as recited in claim 1 wherein step c) includes reconstructing the image by computing a marginalized median of the posterior probability distribution.

4. The method as recited in claim 1 wherein step c) includes reconstructing the image by computing a marginalized maximum of the posterior probability distribution.

5. The method as recited in claim 1 wherein step b) includes using a Markov Chain Monte Carlo technique to estimate sample values of the posterior probability distribution.

6. The method as recited in claim 1 wherein step c) includes reconstructing an image having voxels that contain attenuation-corrected estimates of a number of emissions per voxel.

7. The method as recited in claim 6, wherein step c) further includes producing from the image having voxels that contain attenuation-corrected estimates of the number of emissions per voxel, an image having voxels that contain attenuation-corrected estimates of activity.

8. A method for producing an attenuation map from time-of-flight (“TOF”) positron emission tomography (“PET”) data acquired with a PET system, the steps of the method comprising:
a) acquiring TOF-PET data using the PET system;
b) estimating from the TOF-PET data, a posterior probability distribution of attenuation values; and
c) producing an attenuation map from the posterior probability distribution of attenuation values.

9. The method as recited in claim 8 wherein step c) includes producing the attenuation map by computing a mean of the posterior probability distribution.

10. The method as recited in claim 8 wherein step c) includes producing the attenuation map by computing a marginalized median of the posterior probability distribution.

11. The method as recited in claim 8 wherein step c) includes producing the attenuation map by computing a marginalized maximum of the posterior probability distribution.

12. The method as recited in claim 8 wherein step b) includes using a Markov Chain Monte Carlo technique to estimate sample values of the posterior probability distribution.

13. The method as recited in claim 8 further comprising reconstructing an attenuation-corrected image from the TOF-PET data using the attenuation map produced in step c).

14. A positron emission tomography (“PET”) system for producing attenuation-corrected images of a subject, the PET system comprising:
a plurality of detectors arranged about a bore configured to receive the subject, the plurality of detectors configured to acquire gamma rays emitted from the subject as a result of a radiotracer administered to the subject;
a data acquisition processor in communication with the plurality of detectors and configured to acquire TOF-PET data corresponding to acquired gamma rays;
an image reconstruction processor configured to process the acquired TOF-PET data with steps comprising:
i) estimating from the TOF-PET data, a posterior probability distribution of attenuation-corrected TOF-PET data; and
ii) reconstructing an attenuation-corrected image from the posterior probability distribution of attenuation-corrected TOF-PET data.

15. The system of claim 14, wherein the image reconstruction processor is further configured to apply a Markov Chain Monte Carlo technique to estimate sample values of the posterior probability distribution.

16. The system of claim 14, wherein the image reconstruction processor is further configured to produce an attenuation map by computing one of a mean of the posterior probability distribution, or a marginalized median of the posterior probability distribution, or a marginalized maximum of the posterior probability distribution.

17. The system of claim 16, wherein reconstructing the attenuation-corrected image from the TOF-PET data includes using the attenuation map.

18. The system of claim 14, wherein the image reconstruction processor is further configured to reconstruct an image having voxels that contain attenuation-corrected estimates of a number of emissions per voxel.

19. The system of claim 18, wherein the image reconstruction processor is further configured to produce from the image having voxels that contain attenuation-corrected estimates of the number of emissions per voxel, an image having voxels that contain attenuation-corrected estimates of activity.
